(19) United States
(12) Patent Application Publication
(10) Pub. No.: US 2016/0068826 A1
(43) Pub. Date: Mar. 10, 2016

(54) PEROXIDASES HAVING ACTIVITY FOR CAROTENOIDS

(71) Applicant: Henkel AG & Co. KGaA, Duesseldorf (DE)

(72) Inventors: Thomas Weber, Dormagen (DE); Marion Merkel, Koeln (DE); Inken Prueser, Duesseldorf (DE); Nina Mussmann, Willich (DE); Timothy O'Connell, Duesseldorf (DE); Hendrik Hellmuth, Duesseldorf (DE); Diana Linke, Rehburg (DE); Ralf G. Berger, Hannover (DE)

(21) Appl. No.: 14/946,243

(22) Filed: Nov. 19, 2015

Related U.S. Application Data
(63) Continuation of application No. PCT/EP2014/059916, filed on May 15, 2014.

(30) Foreign Application Priority Data
May 23, 2013 (DE) 10 2013 209 545.7

(51) Int. Cl. C12N 9/08; C11D 3/386

(52) U.S. Cl. CPC C12N 9/0065; C11D 3/38654; C12Y 111/01

(57) ABSTRACT

The invention relates to peroxidases having activity for carotenoids and comprising an amino acid sequence that has at least 70% sequence identity to the amino acid sequence specified in SEQ ID NO:1 across its entire length, washing and cleaning agents that contain such peroxidases and the use thereof.

### FIELD OF THE INVENTION

The present invention generally relates to enzyme technology. The invention relates to peroxidases with activity for carotenoids, the production thereof, all sufficiently similar peroxidases and nucleic acids coding for them, and host organisms containing said nucleic acids. The invention further relates to methods using said peroxidases and to agents containing them, particularly detergents and cleaning agents.

### BACKGROUND OF THE INVENTION

The use of enzymes in detergents and cleaning agents is established in the state of the art. They are used to expand the range of products for the agents in question according to their special activities. These include in particular hydrolytic enzymes such as proteases, amylases, lipases, and cellulases. The first three enzymes mentioned hydrolyze proteins, starches, and fats and therefore contribute directly to soil removal. Cellulases are used in particular because of their effect of fabrics. To increase the bleaching effect, however, oxidoreductases, for example, oxidases, oxygenases, catalases (which react as peroxidases at low H₂O₂ concentrations), peroxidases such as halo-, chloro-, or bromoperoxidases or lignin, glucose, or manganese peroxidases, dioxygenases, or laccases (phenol oxidases, polyphenol oxidases) are also used in the detergents and cleaning agents.

Suitable enzymatic bleaching systems are known in the state of the art, for example, from the international patent publications WO 98/45398 A1, WO 2004/058955 A2, WO 2005/124012, and WO 2005/056782 A2. Such enzymatic systems can be combined advantageously with organic, especially preferably aromatic compounds, interacting with the enzymes, in order to enhance the activity of the oxidoreductases in question (enhancers) or in the case of very different redox potentials to assure the electron flow between the oxidizing enzymes and the soil (mediators).

Conventional bleaching systems with a percarbonate, peroxide, or chlorine base cannot be used in water-containing formulations, i.e., especially many liquid formulations. Moreover, the use of such systems by consumers is perceived as aggressive and harmful to the environment in comparison with enzymatic systems. In this respect, the use of enzymatic systems is desirable for reasons of sustainability.

Enzymatic bleaching systems, however, are typically also based on the enzymatic generation of hydrogen peroxide by the breakdown of suitable enzyme substrates. These substrates must be added to the detergent or cleaning agents and represent an additional cost factor and in part also an additional toxicological or allergological risk factor. Liquid one-component systems furthermore have the problem that the substrate and enzyme come into contact even before use in the washing or cleaning liquor and therefore a premature breakdown of the substrate must be avoided in a costly manner.

Bleaching systems are necessary, however, for removing certain highly staining soils on textiles and hard surfaces, for example, carotenoid-containing soils, in order to achieve a satisfactory cleaning performance. Carotenoid-containing soils on textiles are difficult to remove with conventional liquid detergents. Moreover, carotenoid-containing soils on dishes pose the problem that they are distributed in the cleaning liquor in automatic dishwashing and diffuse into plastics and discolor them. These discolorations are familiar to the user and it is desirable to reduce them.

There is a need, therefore, for substrate-independent enzymatic systems that have a lightening effect particularly on carotenoid-containing soils or decolorize these soils, and prevent the reattachment of staining soils from the washing or cleaning liquor to the items to be washed.

In order to be suitable for use in detergent and cleaning agents, it is desirable, further, that such enzyme systems have an enzymatic activity in the neutral to slightly alkaline pH range and over a broad temperature range up to 95° C., particularly in the range of 30-65° C.

A peroxidase from Bjerkandera adusta has now been found that has the desired properties and therefore is especially highly suitable for use in detergents and cleaning agents. The found peroxidase has a marked activity for carotenoids. As a result, the enzyme can also be used without additional substrates for lightening carotenoid-containing soils.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A peroxidase comprising an amino acid sequence that has at least 98.5%, preferably at least 99% sequence identity to the amino acid sequence set forth in SEQ ID NO:1 over the total length thereof.

An agent, particularly a detergent or cleaning agent, characterized in that it contains at least one peroxidase comprising an amino acid sequence, which has at least 70%, at least 75%, at least 80%, at least 85%, at least 90%, at least 95%, at least 96%, at least 97%, at least 98%, at least 98.5%, or at least 99% sequence identity to the amino acid sequence set forth in SEQ ID NO:1 over the entire length thereof.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In a first aspect, the invention relates to a peroxidase comprising an amino acid sequence that has a sequence identity of at least 98.5%, primarily at least 99%, preferably at least 99.2%, at least 99.3%, at least 99.4%, at least 99.5%, preferably at least 99.6%, at least 99.7%, and especially at least 99.8%, to the amino acid sequence set forth in SEQ ID NO:1 over the entire length thereof. In different embodiments, the peroxidase has an enzymatic activity for carotenoids.

The enzymes described herein are preferably of fungal origin, particularly homologues of the Bjerkandera adusta peroxidase with the amino acid sequence set forth in SEQ ID NO:1.

The peroxidases described herein have an enzymatic activity; in other words, they are capable of cleaving carotenoids oxidatively. The oxidative cleavage is independent of the presence of hydrogen peroxide, but can be enhanced by the presence of hydrogen peroxide. A peroxidase described herein is preferably a mature peroxidase, i.e., the catalytically active molecule without signal peptides and/or propeptide(s). Unless otherwise stated, the provided sequences also refer to mature enzymes in each case.

The term “carotenoids,” as employed herein, relates to compounds from the class of substances of terpenes, which occur as natural dyes producing a yellow to reddish color. About 800 different carotenoids are known, which occur primarily in the chromoplasts and plastids of plants, in bacteria, but also in the skin, shell, and in the carapace of animals and in the feathers and in the egg yolk of birds, if the animal in question consumes dye-containing plant material with its food. Only bacteria, plants, and fungi are capable of synthesizing these pigments de novo. Carotenoids are formally made up of 8 isoprene units and therefore are considered to be tetraterpenes. They are divided into carotenes, which are made up of only carbon and hydrogen, and xanthophylls, i.e., oxygen-containing derivatives of the carotenes. The absorption spectrum of the carotenoids occurs at wavelengths in the range of 400 to 500 nanometers. The best-known and most frequently occurring carotenoid is β-carotene (carrot), which is also known as provitamin A. Other frequently occurring carotenoids are α-carotene, lycopene (tomato), β-cryptoxanthin, capsanthin (red paprika), lutein, and zeaxanthin.

Further, preferred embodiments of the peroxidases have a particular stability in detergents or cleaning agents, for example, to surfactants and/or bleaching agents and/or to temperature effects, especially to high temperatures, especially during the washing or cleaning process, for example, between 50 and 65° C., particularly 60° C., and/or to acidic or alkaline conditions and/or to changes in pH and/or to denaturing or oxidizing agents and/or to proteolytic degradation and/or to a change in the redox conditions.

According to a further special embodiment, the peroxidases according to the invention have a good storage stability in detergents or cleaning agent formulations, for example, measured at 30° C. and higher temperatures, particularly at 35° C. or 40° C.

In different embodiments of the invention, the peroxidase comprises an amino acid sequence, which is at least 98.5%, 98.8%, or 99.0% identical to the amino acid sequence set forth in SEQ ID NO:1 over the entire length thereof. Numerical values, given herein without decimal places, refer in each case to the full given value with a decimal place. Thus, for example, “99%” stands for “99.0%.” The term “approximately” in relation to a numerical value refers to a variation of ±10% with regard to the given numerical value.

In a further aspect, the invention relates to an agent which is characterized in that it contains a peroxidase comprising an amino acid sequence that has a sequence identity of at least 70%, at least 75%, at least 80%, at least 85%, at least 90%, at least 95%, at least 96%, at least 97%, at least 98%, at least 98.5%, or at least 99.0%, preferably at least 99.2%, at least 99.3%, at least 99.4%, at least 99.5%, preferably at least 99.6%, at least 99.7%, or at least 99.8% to the amino acid sequence set forth in SEQ ID NO:1 over the entire length thereof. Such a peroxidase has an enzymatic activity for carotenoids, i.e., is capable of utilizing carotenoids as a substrate and to cleave them oxidatively. The activity for carotenoids is detectable, insofar as it can be measured, for example, using the assay described in the examples and preferably is also quantifiable.

The enzymes used in the agent are preferably of fungal origin, particularly from basidiomycetes, especially preferably homologues of the Bjerkandera adusta peroxidase with the amino acid sequence set forth in SEQ ID NO:1. Preferably, the agent is a detergent or cleaning agent including an (automatic) dishwashing detergent. Because peroxidases described herein have advantageous cleaning performances particularly for carotenoid-containing soils, the agents are suitable and advantageous particularly for removing such carotenoid-containing soils. Such agents contain the peroxidases described herein in an amount from 1×10⁻⁸ to 1% by weight, 1×10⁻⁷ to 0.5% by weight, from 0.00001 to 0.3% by weight, from 0.0001 to 0.2% by weight, and especially preferably from 0.001 to 0.1% by weight, in each case based on the active protein.

The (active) protein concentration can be determined with the use of known methods, for example, the BCA assay (bicinchoninic acid; 2,2′-biquinolyl-4,4′-dicarboxylic acid) or the biuret assay.

The identity of nucleic acid or amino acid sequences is determined by a sequence comparison. This sequence comparison is based on the typically used BLAST algorithm, established in the existing art, (cf, for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pp. 3389-3402) and is carried out basically in that similar sequences of nucleotides or amino acids in the nucleic acid or amino acid sequences are matched to one another. A tabular matching of the positions in question is called an alignment. A further algorithm available in the existing art is the FASTA algorithm. Sequence comparisons (alignments), particularly multiple sequence comparisons, are compiled using computer programs. For example, the Clustal series (cf. for example, Chenna et al. (2003): Multiple sequence alignment with the Clustal series of programs. Nucleic Acid Research 31, 3497-3500), T-Coffee (cf., for example, Notredame et al. (2000): T-Coffee: A novel method for multiple sequence alignments. J. Mol. Biol. 302, 205-217), or programs, based on these programs or algorithms, are frequently used. In the present patent application, all sequence comparisons (alignments) were created using the computer program Vector NTI® Suite 10.3 (Invitrogen Corporation, 1600 Faraday Avenue, Carlsbad, Calif., USA) with the predefined standard parameters, whose AlignX module for the sequence comparisons is based on ClustalW.

A comparison of this kind also permits a conclusion on the similarity of the compared sequences. It is usually given as a percent identity, i.e., the proportion of identical nucleotides or amino acid residues at the same positions or at positions corresponding to one another in an alignment. The more broadly construed term of homology includes conserved amino acid exchanges in the case of amino acid sequences, therefore amino acids with similar chemical activity, because they perform mostly similar chemical activities within the protein. The similarity of compared sequences can therefore also be given as a percent homology or percent similarity. Indications of identity and/or homology can be found for entire polypeptides or genes or only over individual regions. Homologous or identical regions of various nucleic acid or amino acid sequences are therefore defined by matches in the sequences. Such regions often have identical functions. They can be small and comprise only a few nucleotides or amino acids. Often such small regions carry out functions essential for the overall activity of the protein. It can be useful, therefore, to relate sequence matches only to individual, optionally small regions. Unless otherwise stated, the identity or homology data in the present application, however, relate to the entire length of the nucleic acid or amino acid sequence given in each case.

The peroxidases described herein may have amino acid modifications, particularly amino acid substitutions, insertions, or deletions in comparison with the sequence set forth in SEQ ID NO:1. Such peroxidases are developed further, for example, by targeted genetic modifications, i.e., by mutagenesis methods, and optimized for specific purposes or with regard to special properties (for example, with regard to their catalytic activity, stability, etc.). In particular, in addition the pro ducibility, processing, secretion, and other production steps including downstream processing can also be improved by targeted genetic modifications, particularly by mutagenesis methods.

Further, nucleic acids described herein can be introduced into recombination batches and thereby used to generate completely novel peroxidases or other polypeptides.

The aim is to introduce targeted mutations such as substitutions, insertions, or deletions into the molecules in order to improve, for example, the performance of the enzymes described herein. To this end, in particular the surface charges and/or the isoelectric point of the molecules and thereby their interactions with the substrate can be modified. Thus, for example, the net charge of the enzymes can be modified in order to influence thereby the substrate bonding, particularly for use in detergents and cleaning agents. Alternatively or in addition, the stability of the peroxidases can be increased by one or more appropriate mutations and their performance can be improved as a result.

A further object of the invention therefore is a peroxidase, which is characterized in that it can be obtained from a peroxidase as described above as the parent molecule by a single or multiple conservative amino acid substitution. The term “conservative amino acid substitution” means the exchange (substitution) of one amino acid residue for another amino acid residue, whereby this exchange does not lead to a change in the polarity or charge at the position of the exchanged amino acid, e.g., the exchange of one nonpolar amino acid residue for another nonpolar amino acid residue. Conservative amino acid substitutions within the scope of the invention comprise, for example: G=A=S, l=V=L=M, D=E, N=Q, K=R, Y=F, S=T, G=A=I=V=L=M=Y=F=W=P=S=T.

Alternatively or in addition, the peroxidase is characterized in that it can be obtained from a peroxidase described herein as the parent molecule by fragmentation, fusion, deletion, insertion, or substitution mutagenesis and comprises an amino acid sequence that is homologous to the parent molecule over a length of at least 100, 150, 200, 250, 300, 310, 320, 330, 340, 350, 360, or 365 contiguous amino acids.

Proteins obtained by insertion mutation are to be understood as variants that have been obtained by methods known per se by insertion of a nucleic acid or protein fragment into the starting sequences. Because of their basic similarity they may be classified as chimeric proteins. They differ from them only in the relationship between the size of the unmodified protein portion to the size of the entire protein. The proportion of foreign protein in such insertion-mutated proteins is smaller than in chimeric proteins.

Fragments are understood to be all proteins or peptides that are smaller than the natural proteins or those that correspond to completely translated genes and can also be obtained, for example, synthetically. Because of their amino acid sequences, they can be associated with the relevant complete proteins. They can assume similar structures, for example, or perform proteolytic activities or partial activities, such as, for example, the complexing of the substrate. Fragments and deletion variants of parent proteins are similar in principle; whereas fragments tend to represent smaller pieces, only short regions tend to be lacking in deletion mutants (perhaps only one or more amino acids). It is thus possible, for example, to delete other individual amino acids, particularly 1, up to 2, up to 3, up to 4, up to 5, particularly up to 10, preferably up to 20 amino acids at the termini or in the loops of the enzyme, without the enzymatic activity being lost or reduced thereby. Deletions of particularly 1, up to 2, up to 3, up to 4, up to 5, particularly up to 10, preferably up to 20 amino acids on the N-terminal side of the enzyme and/or 1, up to 2, up to 3, up to 4, up to 5, preferably up to 6 amino acids on the C-terminal side of the enzyme are especially preferred. In this regard, the enzymatic activity of the peroxidase is preferably not reduced or reduced only to a limited extent, particularly only up to a reduction of 15% of the activity of the peroxidase according to SEQ ID NO:1.

Further, for example, the allergenicity as well of the enzymes in question can be reduced and thus their usability overall improved by such fragmentation and deletion, fusion, insertion, or substitution mutagenesis. Advantageously, the enzymes retain their enzymatic activity after mutagenesis as well; i.e., their enzymatic activity corresponds at least to that of the parent enzyme; i.e., in a preferred embodiment, the enzymatic activity constitutes at least 80%, preferably at least 90% of the activity of the parent enzyme. Substitutions can also exhibit advantageous effects. Both individual and multiple contiguous amino acids can be exchanged for other amino acids.

Chimeric or hybrid proteins within the meaning of the present application are to be understood as proteins that are composed of elements that originate naturally from different polypeptide chains from the same organism or from different organisms. This procedure is also called shuffling or fusion mutagenesis. The purpose of such a fusion can be, for example, to bring about or to modify a specific enzymatic function with the aid of the fused-on protein part. Within the meaning of the present invention, it is immaterial whether such a chimeric protein consists of a single polypeptide chain or multiple subunits over which different functions can be distributed. To realize the last-mentioned alternative, it is possible, for example, to break down a single chimeric polypeptide chain into a plurality of chains post-translationally or only after a purification step by targeted proteolytic cleavage. In particular, fusion mutagenesis can also be used so that in this regard in a manner known to the skilled artisan a fusion protein is produced that has a part that can be obtained starting from a peroxidase described herein as the parent molecule by fragmentation or deletion, insertion, or substitution mutagenesis and comprises an amino acid sequence that is homologous to the parent molecule over a length of at least 100, 150, 200, 250, 300, 310, 320, 330, 340, 350, 360, or 365 contiguous amino acids and contains another part of a different protein. Preferably, a sequence of one or more different proteins is attached at the C-terminus and/or at the N-terminus of the part derived from the peroxidase.

For example, it is suitable to introduce additional domains, preferably from other proteins that positively influence expression, folding, attachment to specific substrates, or secretion. In particular, a carbohydrate binding domain (CBD) can be inserted in a manner known to the skilled artisan as additional domains at the C-terminus and/or at the N-terminus of the part derived from the peroxidase. Suitable domains are described in the review article of Boraston, A. B., et al., Biochem. J. (2004), 382, pp. 769-781, the content of which is hereby incorporated by reference in its entirety. Such a modification because of improved targeting in textiles with a proportion of cotton leads to an improved cleaning performance in regard to protein-containing spots.

A further subject of the invention is a previously described peroxidase, which is stabilized in addition, particularly by one or more mutations, for example, substitutions, or by linking to a polymer. An increase in the stability during storage and/or during use, for example, in the washing process, then has the result that the enzymatic activity lasts longer and the cleaning performance is thereby improved. Basically all expedient stabilization options and/or those described in the existing art may be used. Preferred stabilizations are those that are achieved by mutations of the enzyme itself, because such stabilizations require no further work steps after the recovery of the enzyme.

Further options for stabilization are, for example:
Modifying the binding of metal ions or cofactors, for example, by exchanging one or more amino acid(s) involved in the binding for one or more other amino acids;
Protecting from the effect of denaturing agents such as surfactants by mutations, which cause a change in the amino acid sequence on or to the surface of the protein;
Exchanging amino acids, which are close to the N-terminus, for those that presumably come into contact via noncovalent interactions with the rest of the molecule and thus contribute to the retention of the globular structure.

Preferred embodiments are those in which the enzyme is stabilized in multiple ways, because multiple stabilizing mutations act additively or synergistically. The enzymes described herein can contain manganese ions as cofactors and are thus stabilized variants, inter alia, those in which the binding of the manganese has been modified.

A further subject of the invention is a peroxidase as described above, which is characterized in that it has at least one chemical modification. A peroxidase with such a modification is designated as a derivative; i.e., the peroxidase is derivatized.

Derivatives within the meaning of the present application accordingly are understood as proteins whose pure amino acid chain has been chemically modified. Such derivatizations can be performed, for example, in vivo by the host cell expressing the protein. Linkages of low-molecular-weight compounds, such as of lipids or oligosaccharides, are to be emphasized in particular in this regard. Derivatizations can also be carried out, however, in vitro, for instance, by chemical conversion of a side chain of an amino acid or by covalent bonding of a different compound to the protein. Linkage of amines to carboxyl groups of an enzyme in order to modify the isoelectric point is possible, for example. Another such compound can also be a further protein that is bound, for example, via bifunctional chemical compounds to a protein described herein. Derivatization is likewise to be understood as covalent bonding to a macromolecular carrier, or also as a noncovalent inclusion into suitable macromolecular cage structures. Derivatizations, for example, can influence the substrate specificity or strength of bonding to the substrate, or can bring about a temporary blockage of enzymatic activity if the linked-on substance is an inhibitor. This can be useful, for example, for the period of storage. Modifications of this kind can furthermore influence stability or enzymatic activity. They can moreover also serve to decrease the allergenicity and/or immunogenicity of the protein and thereby, for example, to increase its skin compatibility. For example, linkages to macromolecular compounds, for example, polyethylene glycol, can improve the protein with regard to stability and/or skin compatibility.

Derivatives of a protein described herein can also be understood in the broadest sense as preparations of said proteins. Depending on recovery, processing, or preparation, a protein can be associated with a variety of other substances, for example, from the culture of the producing microorganisms. A protein can also have had other substances deliberately added to it, for example, in order to increase its storage stability. For this reason, all preparations of a protein described herein are also included. This is also irrespective of whether or not it actually displays this enzymatic activity in a specific preparation. It may be desirable for it to possess little or no activity during storage and to perform its enzymatic function only at the time of use. This can be controlled, for example, by suitable accompanying substances.

The present invention comprises the above-described peroxidases and variants and derivatives thereof both as such and also as a component of an agent, particularly a detergent and cleaning agent, as defined above.

A further subject of the invention is a nucleic acid that codes for a peroxidase described herein, particularly a nucleic acid comprising the nucleotide sequence set forth in SEQ ID NO:2, as well as a vector containing such a nucleic acid, particularly a cloning vector or an expression vector.

These can be DNA molecules or RNA molecules. They can exist as a single strand, as a single strand complementary to said single strand, or as a double strand. In the case of DNA molecules in particular, the sequences of both complementary strands in all three possible reading frames are to be considered in each case. It must be considered further that different codons, therefore, base triplets, can code for the same amino acids, so that a specific amino acid sequence can be coded by multiple different nucleic acids. Because of this degeneracy of the genetic code, all nucleic acid sequences that can encode one of the above-described peroxidases are included in this subject of the invention. The skilled artisan is capable of unequivocally determining these nucleic acid sequences, because despite the degeneracy of the genetic code, defined amino acids are to be associated with individual codons. The skilled artisan, proceeding from an amino acid sequence, can therefore readily ascertain nucleic acids coding for said amino acid sequence. Furthermore, in the case of nucleic acids described herein one or more codons can be replaced by synonymous codons. This aspect refers in particular to heterologous expression of the enzymes described herein. For example, every organism, for example, a host cell of a production strain, possesses a specific codon usage. Codon usage is understood as the translation of the genetic code into amino acids by the respective organism. Bottlenecks in protein biosynthesis can occur if the codons located on the nucleic acid are faced in the organism with a comparatively small number of loaded tRNA molecules. Although it codes for the same amino acid, the result is that a codon is translated less efficiently in the organism than a synonymous codon coding for the same amino acid. Because of the presence of a larger number of tRNA molecules for the synonymous codon, the latter can be translated more efficiently in the organism. Accordingly the present invention also comprises nucleotide sequences that are codon-optimized for expression in a specific host organism. The sequence identity in this regard can be low in comparison with the original, whereby the coded protein remains identical, however.

Using methods commonly known today such as, for example, chemical synthesis or the polymerase chain reaction (PCR) in combination with standard methods of molecular biology or protein chemistry, a skilled artisan is capable of preparing, on the basis of known DNA sequences and/or amino acid sequences, the corresponding nucleic acids up to complete genes.

Vectors within the meaning of the present invention are understood as elements, made up of nucleic acids and containing a nucleic acid described herein as a characterizing nucleic acid region. They make it possible to establish said nucleic acid as a stable genetic element in a species or a cell line over multiple generations or cell divisions. In particular when used in bacteria, vectors are special plasmids, therefore, circular genetic elements. Within the scope of the present invention, a nucleic acid described herein is cloned into a vector. The vectors include, for example, those originating from bacterial plasmids, viruses, or bacteriophages, or predominantly synthetic vectors or plasmids having elements of very diverse origin. With the further genetic elements present in each case, vectors are capable of establishing themselves as stable units in the relevant host cells over multiple generations. They can be present extrachromosomally as separate units or be integrated into a chromosome or into chromosomal DNA

Expression vectors comprise nucleic acid sequences that enable them to replicate in the host cells containing them, preferably microorganisms, especially preferably bacteria, and to express a nucleic acid contained therein. The expression is influenced in particular by the promoter(s) that regulate transcription. In principle, the expression can occur by the natural promoter, originally localized before the nucleic acid to be expressed, but also by a host cell promoter provided on the expression vector or by a modified or completely different promoter of a different organism or a different host cell. In the present case, at least one promoter is provided for the expression of a nucleic acid described herein and used for the expression thereof. Expression vectors can furthermore be regulatable, for example, by a change in culturing conditions or when the host cells containing them reach a specific cell density, or by the addition of specific substances, in particular activators of gene expression. One example of such a substance is the galactose derivative isopropyl-β-D-thiogalactopyranoside (IPTG), which is used as an activator of the bacterial lactose operon (lac operon). In contrast to expression vectors, the contained nucleic acid is not expressed in cloning vectors.

A further subject of the invention is a nonhuman host cell that contains a nucleic acid described herein or a vector described herein, or that contains a peroxidase described herein, in particular one secreting the peroxidase into the medium surrounding the host cell. A nucleic acid described herein or a vector described herein is preferably transformed into a microorganism, which then represents a host cell. The nucleic acid described herein is preferably heterologous in regard to the host organism, i.e., is not a sequence occurring naturally in the host organism. Alternatively, individual components, i.e., nucleic acid parts or fragments of a nucleic acid described herein, can be also be introduced into a host cell in such a way that the then resulting host cell contains a nucleic acid described herein or a vector described herein. This procedure is especially suitable when the host cell already contains one or more constituents of a nucleic acid described herein or a vector described herein, and the further constituents are then correspondingly supplemented. Cell transformation methods are established in the existing art and are sufficiently known to the skilled artisan. All cells are suitable in principle as host cells, i.e., prokaryotic or eukaryotic cells. Host cells that can be advantageously manipulated genetically, for example, as regards the transformation using the nucleic acid or vector and the stable establishment thereof, are preferred, for example, single-celled fungi or bacteria. Further, preferred host cells are notable for being readily manipulated in microbiological and biotechnological terms. This refers, for example, to easy culturability, high growth rates, low demands for fermentation media, and good production and secretion rates for foreign proteins. Preferred host cells described herein secrete the (transgenically) expressed protein into the medium surrounding the host cells. Furthermore, the peroxidases can be modified after their production by cells producing them, for example, by the addition of sugar molecules, formylations, aminations, etc. Post-translational modifications of this kind can functionally influence the peroxidase.

Further embodiments are represented by those host cells whose activity can be regulated on the basis of genetic regulation elements that are provided, for example, on the vector, but can also be present at the outset in these cells. They can be stimulated to expression, for example, by the controlled addition of chemical compounds serving as activators, by modifying the culturing conditions, or when a specific cell density is reached. This makes possible an economic production of the proteins described herein. One example of such a compound is IPTG, as described above.

Host cells can be prokaryotic or bacterial cells. Bacteria are notable for short generation times and few demands in terms of culturing conditions. As a result, cost-effective culturing methods or production methods can be established. In addition, the skilled artisan has a wide range of experience in the case of bacteria in fermentation technology. Gram-negative or Gram-positive bacteria may be suitable for a specific production, for reasons to be determined experimentally in the individual case, such as nutrient sources, product formation rate, time requirement, etc.

In Gram-negative bacteria such as, for example, Escherichia coli, a plurality of proteins are secreted into the periplasmic space, therefore, into the compartment between the two membranes enclosing the cell. This can be advantageous for specific applications. Further, Gram-negative bacteria can also be configured so that they discharge the expressed proteins not only into the periplasmic space but into the medium surrounding the bacterium. Gram-positive bacteria, on the other hand, such as, for example, bacilli or actinomycetes, or other representatives of the Actinomycetales, possess no external membrane, so that secreted proteins are delivered immediately into the medium, as a rule the nutrient medium, surrounding the bacteria, from which medium the expressed proteins can be purified. They can be isolated directly from the medium or processed further. In addition, Gram-positive bacteria are related or identical to most source organisms for technically important enzymes, and usually themselves form comparable enzymes, so that they possess similar codon usage and their protein synthesis apparatus is naturally correspondingly directed.

Host cells described herein can be modified in terms of their requirements for culture conditions, can comprise other or additional selection markers, or can also express other or additional proteins. They can also be, in particular, host cells that transgenically express multiple proteins or enzymes.

The present invention can be used in principle for all microorganisms, in particular for all fermentable microorganisms, and has the result that proteins described herein can be produced by the use of such microorganisms. Such microorganisms then represent host cell within the meaning of the invention.

In a further embodiment of the invention, the host cell is characterized in that it is a bacterium, preferably one that is selected from the group of the genera Escherichia, Klebsiella, Bacillus, Staphylococcus, Corynebacterium, Arthrobacter, Streptomyces, Stenotrophomonas, and Pseudomonas, more preferably one that is selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor, and Stenotrophomonas maltophilia.

The host cell can also be a eukaryotic cell, however, which is characterized in that it possesses a cell nucleus. A further subject of the invention is therefore a host cell characterized in that it possesses a cell nucleus. In contrast to prokaryotic cells, eukaryotic cells are capable of post-translationally modifying the formed protein. Examples thereof are fungi such as basidiomycetes, actinomycetes, or yeasts such as Saccharomyces, Pichia, Hansenula, or Kluyveromyces. This may be especially advantageous, for example, if the proteins are to undergo specific modifications, enabled by such systems, in connection with their synthesis. Modifications that eukaryotic systems carry out particularly in conjunction with protein synthesis include, for example, the bonding of low-molecular-weight compounds such as membrane anchors or oligosaccharides. Oligosaccharide modifications of this kind can be desirable, for example, in order to lower the allergenicity of an expressed protein. Co-expression with the enzymes naturally formed by such cells, for example, cellulases or lipases, can also be advantageous. Thermophilic fungal expression systems, for example, can furthermore be particularly suitable for the expression of temperature-resistant proteins or variants. Fungal expression systems are preferred within the scope of the invention.

The host cells are cultured and fermented in a conventional manner, for example, in discontinuous or continuous systems. In the former case, a suitable nutrient medium is inoculated with the host cells, and the product is harvested from the medium after a period of time to be determined experimentally. Continuous fermentations are notable for the achievement of a flow equilibrium in which, over a comparatively long time period, cells die off in part but also regrow, and the formed protein can be removed simultaneously from the medium.

Host cells described herein are preferably used to produce peroxidases described herein. A further subject of the invention therefore is a method for producing a peroxidase, comprising

a) culturing a host cell described herein and
b) isolating the peroxidase from the culture medium or from the host cell.

Said subject of the invention preferably comprises fermentation methods. Fermentation methods are known from the existing art and represent the actual industrial-scale production step, generally followed by a suitable purification method for the produced product, for example, the peroxidase and described herein. All fermentation methods based on a suitable method for producing a peroxidase described herein represent embodiments of said subject of the invention.

Fermentation methods which are characterized in that fermentation is carried out via an inflow strategy are particularly appropriate. In this case, the media constituents consumed during continuous culturing are fed in. Considerable increases both in cell density and in cell mass or dry mass and/or especially in the activity of the peroxidase of interest can be achieved in this way. Further, the fermentation can also be configured so that undesirable metabolic products are filtered out or are neutralized by the addition of a buffer or suitable counterions.

The produced peroxidase can be harvested from the fermentation medium. A fermentation method of this kind is preferred over isolation of the peroxidase from the host cell, i.e., product preparation from the cell mass (dry mass), but requires the provision of suitable host cells or one or more suitable secretion markers or mechanisms and/or transport systems, so that the host cells secrete the peroxidase into the fermentation medium. Alternatively, without secretion, the peroxidase can be isolated from the host cell, i.e., purification thereof from the cell mass, for example, by precipitation using ammonium sulfate or ethanol, or by chromatographic purification.

All the above facts can be combined into methods for producing peroxidases described herein.

In the agents described herein, particularly detergents and cleaning agents, the enzymes to be used can be formulated together with accompanying substances, for instance, from the fermentation, or with stabilizers. In liquid formulations, the enzymes are preferably used as liquid enzyme formulation(s).

The peroxidases can be protected especially during storage from damage such as, for example, inactivation, denaturation, or decomposition, for instance, by physical effects, oxidation, or proteolytic cleavage. Inhibition of proteolysis is especially preferred in microbial production. The described agents may contain stabilizers for this purpose.

Peroxidases with cleaning activity are generally not provided in the form of the pure protein but rather in the form of stabilized, storable, and transportable formulations. These ready-made formulations include, for example, the solid preparations obtained by granulation, extrusion, or lyophilization or, in particular in the case of liquid agents or gel-like agents, solutions of the enzymes, advantageously as concentrated as possible, low in water, and/or combined with stabilizers and other aids.

Alternatively, the enzymes may be encapsulated both for solid and liquid delivery forms, for example, by spray-drying or extrusion of the enzyme solution together with a preferably natural polymer or in the form of capsules, for example, those in which the enzymes are enclosed as in a solidified gel, or in those of the core-shell type, in which an enzyme-containing core is coated with a water-, air-, and/or chemical-impermeable protective layer. In addition, further active ingredients, for example, stabilizers, emulsifiers, pigments, bleaches, or dyes, can be applied in deposited layers. Such capsules are applied by methods known per se, for example, by agitated or roll granulation or in fluidized bed processes. Advantageously, such granules are low-dusting, for example, due to application of polymeric film formers, and storage-stable as a result of said coating.

It is possible, furthermore, to formulate two or more enzymes together, so that a single granule has multiple enzymatic activities.

As is clear from the preceding explanations, the enzyme protein constitutes only a fraction of the total weight of conventional enzyme preparations. Preferably used peroxidase preparations contain between 0.1 and 40% by weight, preferably between 0.2 and 30% by weight, especially preferably between 0.4 and 20% by weight, and in particular between 0.8 and 10% by weight of the enzyme protein.

The agents described herein comprise all conceivable types of detergents or cleaning agents, both concentrates and also agents to be used in undiluted form, for use on a commercial scale, in the washing machine, or washing or cleaning by hand. They include, for example, detergents for textiles, carpets, or natural fibers for which agents the term detergent is used. They also include, for example, dishwashing detergents for dishwashers or manual dishwashing liquids or cleaners for hard surfaces such as metal, glass, porcelain, ceramics, tiles, stone, painted surfaces, plastics, wood, or leather for which the term cleaning agent is used, therefore, in addition to manual and automatic dishwashing agents, for example, also scouring agents, glass cleaners, toilet cleaners, etc. The detergents and cleaning agents within the scope of the invention include further washing additives that are dispensed into the actual detergent in manual or automatic textile laundering in order to achieve a further effect. Further, detergents and cleaning agents within the scope of the invention also include textile pre- and post-treatment agents, therefore, agents with which the laundered item is brought into contact before the actual laundering, for example, in order to loosen stubborn stains, as well as agents that, in a step following the actual textile laundering, impart to the washed item further desirable properties such as a pleasant feel, absence of creases, or low static charge. Fabric softeners, among others, are included among the latter agents.

An agent described herein contains the peroxidase advantageously in an amount from 2 μg to 20 mg, preferably from 5 μg to 17.5 mg, particularly preferably from 20 μg to 15 mg, and very particularly preferably from 50 μg to 10 mg per gram of the agent. Further, the peroxidase, contained in the agent, and/or further ingredients of the agent, can be encased with a substance that is impermeable to the enzyme at room temperature or in the absence of water, which substance becomes permeable to the enzyme under the agent's utilization conditions. Such an embodiment of the invention is thus characterized in that the peroxidase is encased with a substance that is impermeable to the peroxidase at room temperature or in the absence of water. Furthermore, the detergent or cleaning agent itself can also be packaged in a container, preferably an air-permeable container, from which it is released shortly before use or during the washing operation.

These embodiments of the present invention comprise all solid, powdered, liquid, gel-like, or pasty delivery forms of the agents described herein, which optionally can consist of multiple phases and be present in compressed or uncompressed form. The agents can be present as a pourable powder, in particular with a bulk weight from 300 g/L to 1200 g/L, in particular 500 g/L to 900 g/L, or 600 g/L to 850 g/L. The solid delivery forms of the agent include further extrudates, granules, tablets, or pouches. Alternatively, the agent can also be liquid, gel-like, or pasty, for example, in the form of a nonaqueous liquid detergent or dishwashing detergent or a nonaqueous paste or in the form of an aqueous liquid detergent or dishwashing detergent or a hydrous paste. A liquid, gel-like, or pasty agent can be presented in a water-soluble encasement, which dissolves during use of the product in water, for example, heat-sealed in a polyvinyl alcohol film. Furthermore, the agent can be present as a one-component system. Such agents consist of one phase. Alternatively, an agent can also consist of multiple phases. An agent of this kind is thus distributed into multiple components.

Detergents or cleaning agents described herein can contain, in addition to the peroxidase described herein, hydrolytic enzymes or other enzymes as well, in a concentration useful for the effectiveness of the agent. The enzymes can be present in the form of the above-described enzyme formulations. A further embodiment of the invention thus represents agents that moreover comprise one or more further enzymes. All enzymes that can display catalytic activity in the agent described herein are preferably usable as further enzymes, in particular, a protease, amylase, cellulase, hemicellulase, mannanase, tannase, xylanase, xanthanase, xanthan lyase, xyloglucanase, β-glucosidase, pectinase, pectate lyase, carrageenase, perhydrolase, oxidase, oxidoreductase, cutinase, or a lipase, as well as mixtures thereof. Further enzymes are contained in the agent advantageously in an amount in each case from 1×10⁻⁸ to 5% by weight, based on active protein. Increasingly preferably, each further enzyme is contained in the agents described herein in an amount from 1×10⁻⁷ to 3% by weight, from 0.00001 to 1% by weight, from 0.00005 to 0.5% by weight, from 0.0001 to 0.1% by weight, and especially preferably from 0.0001 to 0.05% by weight, based on active protein.

The washing or cleaning agents described herein, which may be present as powdered solids, in recompressed particle form, as homogeneous solutions or suspensions, can contain, apart from a peroxidase described herein, all known ingredients typical in such agents, at least one further ingredient preferably being present in the agent. The agents described herein can contain in particular surfactants, builders, other bleaching agents, or bleach activators. They can contain further water-miscible organic solvents, sequestering agents, electrolytes, pH regulators, and/or further aids such as optical brighteners, graying inhibitors, foam regulators, and dyes and aromatic principles, as well as combinations thereof. In different embodiments of the invention, the agents described herein contain a hydrogen peroxide source, for example, a percarbonate, peroxide, or perborate. The hydrogen peroxide from this source can increase further the catalytic activity of the peroxidases described herein. It is preferable, however, that the enzymes described herein can effect an oxidative cleavage of carotenoids in the absence of hydrogen peroxide as well.

Advantageous ingredients of agents described herein are disclosed in the international patent application WO 2009/121725, beginning therein on page 5, next-to-last paragraph, and ending on page 13 after the second paragraph. Reference is expressly made to this disclosure, and the disclosure content therein is incorporated into the present patent application.

A further subject of the invention is a method for cleaning textiles or hard surfaces which is characterized in that an agent described herein is utilized in at least one method step, or that a peroxidase described herein is catalytically active in at least one method step, in particular in such a way that the peroxidase is used in an amount from 40 μg to 4 g, preferably from 50 μg to 3 g, particularly preferably from 100 μg to 2 g, and very particularly preferably from 200 μg to 1 g.

This includes both manual and automatic methods, automatic methods being preferred. Methods for cleaning textiles are generally notable in that, in multiple method steps, various substances having cleaning activity are applied onto the material to be cleaned and are washed out after the contact time, or that the material to be cleaned is treated in another fashion with a detergent or a solution or a dilution of said agent. The same applies to methods for cleaning all materials other than textiles, particularly hard surfaces. All conceivable washing or cleaning methods can be supplemented, in at least one of the method steps, by the utilization of a detergent or cleaning agent described herein or a peroxidase described herein, and then represent embodiments of the present invention. All facts, subject matters, and embodiments that are described for the peroxidases described herein and agents containing them are also applicable to this subject of the invention. Reference is therefore expressly made at this juncture to the disclosure at the corresponding juncture, with the indication that this disclosure is also valid for the methods described above.

Embodiments of this subject of the invention also represent methods for treating textile raw materials or for textile care, in which a peroxidase described herein becomes active in at least one method step. Preferred here are methods for textile raw materials, fibers, or textiles having natural constituents, and very particularly for those having wool or silk.

A further subject of the invention is the use of an agent described herein for cleaning textiles or hard surfaces, or a peroxidase described herein for cleaning textiles or hard surfaces, particularly such that the peroxidase is used in an amount from 40 μg to 4 g, preferably from 50 μg to 3 g, especially preferably from 100 μg to 2 g, and very especially preferably from 200 μg to 1 g.

A typical starting formulation for a preferably usable automatic dishwashing detergent, for example, in tablet form, comprises the following substances:

| | |
|---|---|
| Na tripolyphosphate | 20-50% by weight |
| Sodium bicarbonate | 10-30% by weight |
| Sodium percarbonate | 5-18% by weight |
| Bleach activator | 0.5 to 5% by weight |
| Bleach catalyst | 0.01-1% by weight |
| Sulfonated polymer | 2.5-15% by weight |
| Polycarboxylate | 0.1-10% by weight |
| Nonionic surfactant | 0.5-10% by weight |
| Phosphonate | 0.5-5% by weight |
| Proteases | 0.1-5% by weight |
| Amylase | 0.1-5% by weight, |

whereby the data in % by weight in each case refer to the entire agent. Instead of the or a part of the tripolyphosphate, in particular 10-50% by weight of citrate or MGDA or GLDA or EDDS or mixtures of two or three of these substances can also be used in the formulation.

All facts, subject matters, and embodiments that are described for the peroxidases described herein and agents containing them are also applicable to this subject of the invention. Reference is therefore expressly made at this juncture to the disclosure at the corresponding juncture, with the indication that this disclosure is also valid for the previously described use.

### EXAMPLES

All molecular biology work steps follow standard methods. Enzymes and kits were used according to the instructions of the particular manufacturer.

### Example 1

### Preparation, Cleaning, and Activity Measurement of a B. adusta Peroxidase

Before use, all media and labware were sterilized (autoclaved). Stock cultures of Bjerkandera adusta were cultured in 2 L of SNL medium (30.0 g L⁻¹ glucose monohydrate; 4.5 g L⁻¹ L-asparagine monohydrate; 1.5 g L⁻¹ KH₂PO₄; 0.5 g L⁻¹ MgSO₄; 3.0 g L⁻¹ yeast extract; 1.0 mL L⁻¹ trace element solution containing 0.005 g L⁻¹ CuSO₄×5 H₂O, 0.08 g L⁻¹ FeCl₃×6 H₂O, 0.09 g L⁻¹ ZnSO₄×7 H₂O, 0.03 g L⁻¹ MnSO₄×H₂O, and 0.4 g L⁻¹ EDTA) with 0.01% defoamer at 24° C., 1 L/min of oxygen, and 200 rpm.

200 mL of a 7-day-old preculture was homogenized by means of an Ultra-Turrax and used for inoculating a main culture. Samples of the supernatant were taken daily and tested for their carotenoid-degrading activity by means of the assay described below. The supernatant was harvested at a time when sufficient activity could be detected and purified by means of anion exchange chromatography. Active fractions were pooled, partially decolorized with activated charcoal, and concentrated by means of ultrafiltration.

Photometric Activity Assay

A supernatant concentrated 10-fold was used for the photometric determination of the carotene-degrading activity. Depending on the activity and the carotenoid used as the substrate, 10-100 μL of the sample and water/buffer were combined to give 1.6 mL. The mixture was heated to 30° C. and the reaction was started by addition of the substrate. The decrease in extinction at the absorption maximum was measured for 10 minutes and the enzyme activity was calculated using the following formula:

A [mU/mL]=ΔE×1.7×1000000×F/(ε×1.6)

ΔE=decrease in extinction at the absorption maximum per minute
ε=extinction coefficient in L×mol⁻¹×cm⁻¹
F=sample dilution factor

The following carotenoids were tested as substrate:

TABLE 1

| | Absorption | Extinction coefficient |
|---|---|---|
| Carotenoid | maximum (nm) | (L × mol⁻¹ × cm⁻¹) |
| Carotene | 460 | 87300 |
| Lycopene | 483 | 114600 |
| Lutein | 457 | 66900 |
| Capsanthin | 478 | 81900 |

Purification of the Peroxidase

A Q-Sepharose column (GE Healthcare, 1 mL) and 20 mM of sodium acetate buffer pH 5 (with and without 1 M sodium chloride) were used for purifying the peroxidase. 10 mL of the supernatant was mixed 1:1 with running buffer. The separation was carried out at a flow rate of 2 mL/min with a linear gradient (running buffer+1 M NaCl) for 15 mL. 1-mL fractions were collected and tested for their enzymatic activity by means of the assay described above.

### Example 2

### Lightening of an Automatic Dishwashing Liquor

As an example for a carotenoid-laden automatic dishwashing detergent (MGSM) dishwashing liquor, 10 g of a commercial tomato brand (concentrated twofold) was brought into contact with an enzyme-containing commercial dishwashing detergent according to Table 2 and deionized water to 200 mL. The pH was adjusted to 9.18 with a sodium hydroxide solution after the dissolving before the final filling to 200 mL.

TABLE 2
Composition of the automatic dishwashing detergent

| | Base |
|---|---|
| Phosphate (% by weight) | 35.9 |
| Sodium carbonate (% by weight) | 12.2 |
| Phosphonate (% by weight) | 2.4 |
| Sulfonic acid group-containing polymer (% by weight) | 7.9 |
| Polyacrylate (% by weight) | 4.6 |
| Nonionic surfactants (% by weight) | 6.1 |
| Percarbonate (% by weight) | 14.6 |
| TAED (% by weight) | 2.3 |
| Bleach catalyst (% by weight) | 1.0 |
| Polycarboxylate (% by weight) | 1.5 |
| Sodium silicate/polycarboxylate (% by weight) | 3.9 |
| Enzyme composition (amylase) (% by weight) | 1.0 |
| Zinc acetate (% by weight) | 0.2 |
| Remainder (perfume, colorants, protease, or protease | To 100 |
| mixture, etc.) (% by weight) | |

Two batches were tested:

Enzyme value: 9 ml, of the above dishwashing liquor with 1 mL of enzyme preparation from Example 1
Blank value: 9 mL of the above dishwashing liquor with 1 mL of deionized water.

Both batches were turned upside down and back, for instance, every second in closed 14-mL polypropylene tubes in a rotary mixer, which was located under a heating mantle heated to 50° C. After 0 hours, 4 hours, 21 hours, and 25 hours a 1-mL sample was taken in each case and cleared of turbid substances in a tabletop centrifuge; the supernatant was used for the extinction measurement against air at 500 nm.

The difference of the enzyme value (the “sample difference”) and the blank value was determined for each time period (negative values indicate a lower extinction of the enzyme value and thereby a lighter sample). The time-dependent “enzymatic lightening” was calculated by forming the difference of the sample difference at time x to the sample difference of the starting value (0 hours) (negative enzymatic lightening values indicate a decrease in the extinction and thereby a greater lightening by the enzyme sample).

TABLE 3

| Time (h): | 0 | 4 | 21 | 25 |
|---|---|---|---|---|
| Enzymatic | 0 | −0.114 | −0.315 | −0.387 |
| lightening | | | | |

The results support a lightening, increasing with time, of the washing liquor by the enzyme.

### Example 3

### Mini Washing Test (Liquid Detergent, Tomato & Carrot)

An enzyme preparation from the culture supernatant of B. adusta with a CDA (carotenoid-degrading activity) of 25 U/L was prepared and used in the following washing test:

Round punched-out soiled samples (diameter of 1 cm) were placed individually in a 48 well microtiter plate (WfK 10O (cotton soiled with carrot juice) and WfK 10SG (cotton soiled with tomato beef sauce).

1000 μL of a washing liquor, preheated to 40° C., of a liquid detergent with the composition given below was pipetted onto each small piece of cloth (end concentration in the test of 4.7 g/L, 16° dH [German degrees of hardness]), and 20 μL of the enzyme solution to be tested was added. The tests were run in triplicate.

A liquid detergent with the following composition was used as the basic detergent formulation (all data in percentage by weight):

0.3-0.5% xanthan, 0.2-0.4% anti-foaming agent, 6-7% glycerol, 0.3-0.5% ethanol, 4-7% FAEOS (fatty alcohol ether sulfate), 24-28% nonionic surfactants, 1% boric acid, 1-2% sodium citrate (dihydrate), 2-4% soda, 14-16% coconut fatty acids, 0.5% HEDP (1-hydroxyethane-1,1-didiphosphonic acid), 0-0.4% PVP (polyvinylpyrrolidone), 0-0.05% optical brightener, 0-0.001% dye, remainder demineralized water.

The plates were closed permeable to air with the associated lid and washed for 1, 4, or 16 hours in the dark on a Titramax incubator shaker at 40° C.

Next, the washing liquor was poured off through a screen and rinsed three times with tap water and three times with deionized water; the remaining water was carefully drawn off by dabbing with lab soakers, and dried for 24 or 48 hours at room temperature in the dark. After the samples were glued to white paper, the lightness and color was measured with a Minolta colorimeter in comparison with the device's white and black standard.

To evaluate the lightening, the difference of the lightness value L* in the L*a*b* system of the enzyme-treated sample to an identically treated, enzyme-free sample (x=0) (averages of the triplicates) was calculated for each soiling (“ΔL*”). The lightening after a 4-hour treatment is presented in the following table (higher values indicate greater lightening of the sample):

TABLE 4

| Fabric | ΔL* |
|---|---|
| WfK 10O | 0.2 |
| WfK 10SG | 4.8 |
| Sum | 5.0 |

The enzyme treatment brings about a considerable lightening compared with the enzyme-free control, which can also be seen with the eye.

Additionally, to evaluate the change in color intensity (decoloration) for another washing test carried out in a similar way, the vector addition of the three color components \sqrt{ΔL*²+Δa*²+Δb*²} was calculated from the differences of the color values ΔL* and in a similar way Δa* and Δb*, between the test batch with the enzyme and the control without the enzyme. As a result, the color shift, highly visible to the eye, from orange-yellow toward color neutrals is reflected better than by the lightening ΔL* alone.

The following table presents the individual contributions by the two tested soils to the sum of the thus calculated decoloration in this experiment (higher values signify better decoloration):

| Decoloration | 1 hour | 4 hours |
|---|---|---|
| WfK 10O | 0.8 | 1.9 |
| WfK 10SG | 4.2 | 11.3 |
| Sum | 5.0 | 13.2 |

The results substantiate an advantageous decoloration of spots by the enzyme. The effect increases with the washing time.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A peroxidase comprising an amino acid sequence that has at least 98.5% sequence identity to the amino acid sequence set forth in SEQ ID NO:1 over the total length thereof.

2. A nucleic acid coding for a peroxidase according to claim 1, comprising the nucleotide sequence set forth in SEQ ID NO:2.

3. A vector containing a nucleic acid according to claim 2.

4. A nonhuman host cell that contains a nucleic acid according to claim 2 or a vector according to claim 3, or that contains a peroxidase according to claim 1.

5. A method for producing a peroxidase comprising
a) culturing a host cell according to claim 4 and
b) isolating the peroxidase from the culture medium or from the host cell.

6. An agent, characterized in that it contains at least one peroxidase comprising an amino acid sequence, which has at least 70% sequence identity to the amino acid sequence set forth in SEQ ID NO:1 over the entire length thereof.

7. The agent according to claim 6, characterized in that
(i) the peroxidase can be obtained from a peroxidase with the amino acid sequence set forth in SEQ ID NO:1 as the parent molecule by single or multiple conservative amino acid substitution; and/or
(ii) the peroxidase can be obtained from a peroxidase with the amino acid sequence set forth in SEQ ID NO:1 as the parent molecule by fragmentation or fusion, deletion, insertion, or substitution mutagenesis and comprises an amino acid sequence that is homologous to the parent molecule over a length of at least 100, 150, 200, 250, 300, 310, 320, 330, 340, 350, 360, or 365 contiguous amino acids.

8. The agent according to claim 6, characterized in that the agent
(i) is a liquid, water-containing detergent or cleaning agent,
(ii) further comprises a hydrogen peroxide source; and
(iii) further comprises surfactants, builders, enzymes different from the peroxidase, bleaching agents, bleach activators, water-miscible organic solvents, sequestering agents, electrolytes, pH regulators, and/or further aids such as optical brighteners, graying inhibitors, foam regulators, and dyes and aromatic principles, as well as combinations thereof.

9. A method for cleaning textiles or hard surfaces, characterized in that an agent according to claim 1 is contacted with textiles or hard surfaces in a wash liquor.


SEQUENCE LISTING (2 sequences)

SEQ ID NO: 1 - protein, 366 aa, Bjerkandera adusta
MAFKQLAAALSIALALPFSQAAITRRVACPDGVNTATNAACCALFAVRDDIQQNLFDGGECGEEVHESLRLTFHDAIGISPSLAATGKFGGGGADGSIMIFDDIEPNFHANNGVDEIINAQKPFVAKHNMTAGDFIQFAGAVGVSNCPGAPQLSFFLGRPAATQPAPDGLVPEPFDSVTDILNRFADAGGFTTQEVVWLLASHSIAAADHVDPTIPGSPFDSTPEIFDTQFFVETLLKGTLFPGTSGNQGEVESPLAGEIRLQSDADFARDSRTACEWQSFVNNQPRMQVLFKAAMQKLSILGHDLTQMIDCSDVIPVPPSTAVRGSHLPAGNTLDDIEQACASTPFPSLTADPGPATSVAPVPPS

SEQ ID NO: 2 - DNA, 1101 bp, Bjerkandera adusta
atggccttcaagcaactcgctgctgctctttccatcgcccttgctctccccttctcgcaagctgcgatcaccagacgtgtggcttgcccagatggcgtgaacaccgcaaccaacgcagcctgttgtgctttgttcgccgtccgtgatgacatccaacagaacttgttcgacggcggcgagtgcggcgaagaagtgcacgagtcacttcgactgaccttccacgatgctattggcatatctccaagccttgccgccactggcaaattcggcggcggaggtgccgacgggtctatcatgatcttcgacgacatcgagcccaacttccacgccaacaacggcgtcgacgagattatcaacgcgcagaagcccttcgtggccaagcacaacatgacggcaggcgactttattcaattcgcaggcgccgttggtgtgagcaactgccctggtgctcctcaactgagcttcttcctcgggcgccctgcagcgacgcagcccgcgcctgacgggcttgttccggagcccttcgactcggtcaccgacatcctcaatcgctttgccgatgctggcggcttcacaacccaagaagttgtttggctccttgcctctcattccattgctgccgctgaccacgtcgacccgacgatccctggatcacccttcgattctactcccgaaatcttcgacacacagttctttgttgagacgttgttgaagggcacgttgttcccaggtacgagcggcaaccagggcgaagtcgagtccccacttgccggcgaaattcgtctccagtcagatgccgacttcgcacgtgactcgaggactgcttgcgagtggcagtctttcgtcaataaccagccccggatgcaagttctgttcaaggcggctatgcagaagctgtctatcttgggccacgatctcactcagatgattgactgctccgacgtaatccctgtacctccgagcacagcggtccgtggatcgcatctgcctgcgggcaacacactggacgacattgaacaggcttgcgcctccacgccattcccctcgctcaccgccgaccctggtccggccacctctgttgcccctgtcccgccttcgtaa